(19) United States
(12) Patent Application Publication
(10) Pub. No.: US 2005/0084395 A1
(43) Pub. Date: Apr. 21, 2005

(54) Vacuum driven pump for a lavage instrument

(76) Inventor: Jing X. Kang, North Andover, MA (US)

Correspondence Address: Melissa Patangia, Lambert & Associates, 92 State Street, 2nd Floor, Boston, MA, 02109, US

(21) Appl. No.: 10/967,485

(22) Filed: Oct. 18, 2004

Related U.S. Application Data
(60) Provisional application No. 60/512,332, filed on Oct. 17, 2003.

(51) Int. Cl. A61N 1/30; F04B 9/08

(52) U.S. Cl. 417/392; 417/390

(57) ABSTRACT

The fluid pump of the present invention is used in a lavage instrument that is driven by vacuum pressure supplied by an external vacuum source. The vacuum driven fluid pump is well suited for lavage instruments that are already connected to an external vacuum source to provide aspiration functions. The present invention is lightweight, inexpensive, disposable, and is driven by a alternative power source.

### CROSS-REFERENCE TO RELATED APPLICATIONS

This application is based on and claims priority from provisional patent application Ser. No. 60/512,332 filed on Oct. 17, 2003.

### FIELD OF THE INVENTION

The present invention relates to a fluid pump used in a lavage instrument.

### BACKGROUND OF INVENTION

In many medical and surgical procedures, irrigating wounds, and surgical areas with fluids and/or removing various irrigating fluids from the surgical area are often necessary. Lavage instruments are well known in the medical arts. Lavage instruments that are connected to an external vacuum source to additionally provide an aspiration function have been developed. Typically, lavage instruments have been connected to external fluid pumps, which supply irrigation fluids to the lavage instrument from an external fluid source, such as a hanging fluid bag. Heretofore, lavage instruments that include internal fluid pumps have been electrically powered, which increases the size, weight and cost of the lavage instrument.

Utility Pat. No. 5,542,918 issued to Atkinson discloses a vacuum driven fluid pump for an aspiration/irrigation instrument. However, the present invention makes significant improvements in apparatus components and functional capabilities. First, the present invention is easy to make and is easy to use. Second, functionally, the present invention can provide continuous fluid flow or at least higher frequency pulsatile fluid flow and is capable of simultaneously aspirating fluid and irrigating fluid.

For these reasons a lavage instrument, which includes an internal fluid pump that is lightweight, inexpensive, disposable and driven by an alternative power source is needed.

### SUMMARY OF THE INVENTION

The fluid pump of this invention is used in a lavage instrument that is driven by vacuum pressure supplied by an external vacuum source. The vacuum driven fluid pump is well suited for lavage instruments that are already connected to an external vacuum source to provide aspiration functions.

These and other features, aspects and advantages of the present invention will become better understood with reference to the following description, claims, and accompanying drawings. Therefore, the form of the invention, as set out above, should be considered illustrative and not as limiting the scope of the following claims.

### BRIEF DESCRIPTION OF DRAWINGS

FIG. 1A is a sectional view of the apparatus of the invention showing the chambers, pistons, valves and their connections, switch, and vacuum controllers;

FIG. 1B is a sectional view and internal layout of the auto-switch of this invention;

FIG. 1C is a sectional view of an alternate embodiment of the apparatus of the invention showing the chambers, pistons, valves and their connections, switch, and vacuum controllers;

FIG. 2 is a sectional view of an alternate embodiment of this invention showing two chambers/pistons set side by side (parallel);

FIG. 3 is a sectional view of an alternate embodiment of this invention showing two different vacuum controllers and without the auto-switch;

FIG. 4 is a sectional view of an alternate embodiment of this invention showing a single chamber with a volume setter (controller);

FIG. 5 is a perspective view of different probes/pipes for aspiration and fluid delivery connected to the pump of the present invention.

### DESCRIPTION OF THE INVENTION

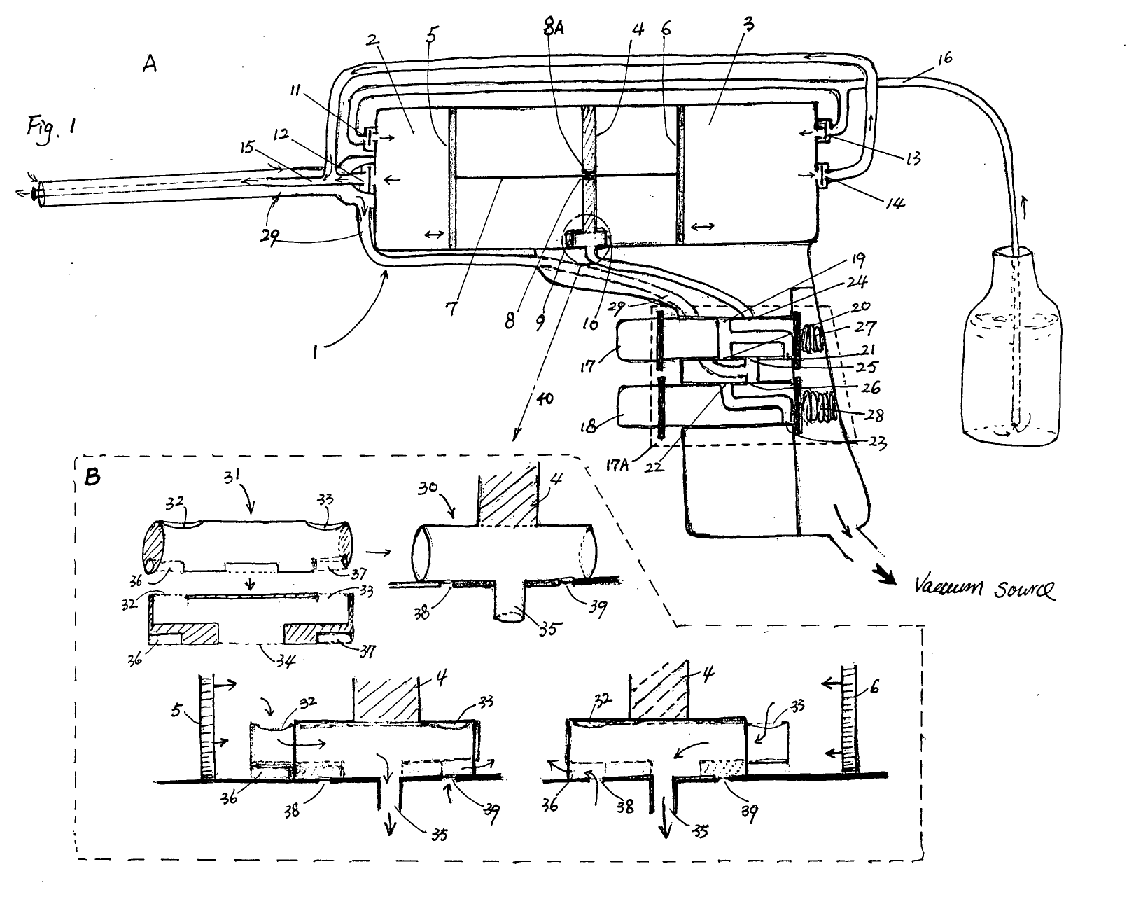

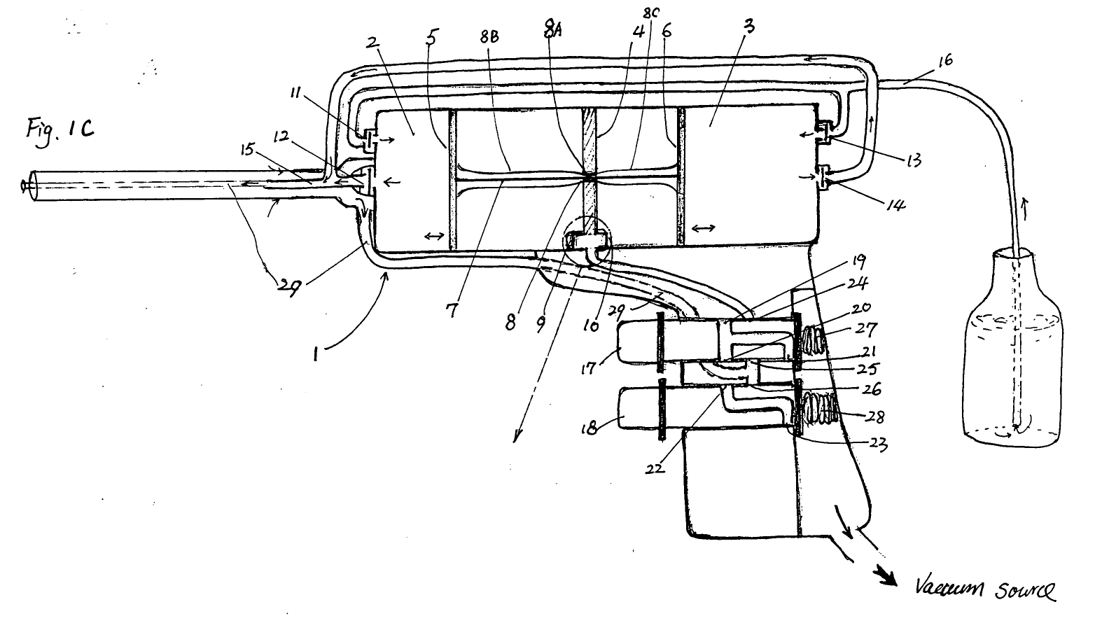

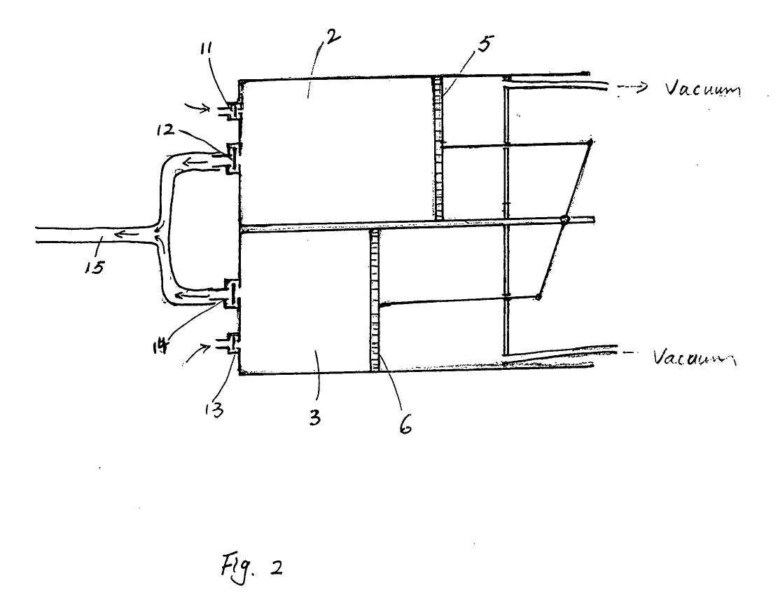

The preferred embodiment of the present invention represents a vacuum driven pump for a lavage instrument as shown in FIGS. 1-5.

With reference to FIG. 1A, FIG. 1A depicts the pump body of the present invention. A cylinder casing 1 has two chambers, the first chamber 2 and the second chamber 3, which are separated by a wall 4 that is lined up end by end. The first and second chambers 2 and 3 each contain a reciprocating piston, the first piston 5 and the second piston 6 respectively. The first and second pistons 5 and 6 are linked by a rod 7, which goes through a hole 8 on the separating wall 4. An elastic cushion 8A is placed around the hole 8 to maintain the disconnection between the first and second chambers 2 and 3, but also allows the free movement of the rod 7. Alternatively, two flexible foldable bags 8B and 8C (depicted in FIG. 1C), one in each of the first and second chambers 2 and 3, are used to enclose the rod 7 and the link between the wall 4 and the first and second pistons 5 and 6. The first and second chambers 2 and 3 each have a port, the first port 9 and the second port 10 respectively, preferably near the separating wall 4, which is connected alternately to the vacuum source or atmosphere. The first and second ports 9 and 10 are located at either side of the first and second pistons 5 and 6.

There are two one-way valves 11, 12, 13, and 14 at each end of the cylinder 1. The first and second chambers 2 and 3 each have two valves: the first and second inlet valves 11 and 13 allow fluid to enter the first and second chambers 2 and 3 respectively; the first and second outlet valves 12 and 14 allow fluid to exit the first and second chambers 2 and 3 respectively.

With further reference to FIG. 1A, the following is a description of the operation of the pump body of the present invention. When vacuum is applied to the first chamber 2 through the first port 9, the second port 10 at the second chamber 3 is now open to atmosphere. The first piston 5 is driven to move from one end of the first chamber 2 to the other end of the first chamber 2. The second piston 6 at the second chamber 3 simultaneously moves. The movement of the first piston 5 in the first chamber 2 with vacuum draws fluid into the first chamber 2 through the first inlet valve 11, while the movement of the second piston 6 in the second chamber 3 expels fluid out of the second chamber 3 through the second outlet valve 14.

When the vacuum switch connects vacuum to the second port 10 in the second chamber 3, the first and second pistons 5 and 6 move in opposite directions. Now, the first chamber 2 expels fluid via the first outlet valve 12, while the second chamber 3 draws fluid via the second inlet valve 13.

If the first and second outlet valves 12 and 14 are connected to the same delivery tube 15, and the first and second inlet valves 11 and 13 are connected to the same liquid source 16, the fluid is able to flow continuously in and out of the cylinder 1.

Further, with reference to FIG. 1A, the following is a description of the vacuum control of the present invention. Two trumpet valves, the top trumpet valve 17 and the bottom trumpet valve 18, are each cylindrical in structure, and each slide into a housing 17A. The top trumpet valve 17 has first, second, and third top holes and channels 19, 20, 21 and the bottom trumpet valve 18 has first and second bottom holes and channels 22 and 23. The top and bottom holes and channels match the corresponding first, second, and third housing holes 24, 25, and 26 on the sidewalls of the housing 17A to communicate to one or two sources. The top and bottom trumpet valves 17 and 18 are able to shuttle between two positions, open or closed, powered by springs, the top and bottom springs 27 and 28.

In the event that both the top and bottom trumpet valves 17 and 18 are utilized, both the top and bottom trumpet valves 17 and 18 are normally in the closed position, disconnected from the vacuum source. The bottom trumpet valve 18 connects the vacuum source to the aspiration pipet 29 when the bottom trumpet valve is pushed down until hole and channel 22 matches hole 26. This configuration is for suction purposes only. The top trumpet valve 17 communicates the vacuum source to both the aspiration pipet 29 and the tube 35 (depicted in FIG. 1B) of the driving force of the first and second pistons 9 and 10 when the top trumpet valve 17 is pushed down until hole and channel 19 matches hole 24, and hole and channel 20 matches hole 25. This configuration is for the purpose of suction and simultaneous sprinkling.

In another configuration, in the event that a single trumpet value is utilized, for example the combination of the top trumpet valve 17 or the bottom trumpet valve 18 into a single trumpet valve, the one trumpet valve to function as above can be operated at two levels. The first level, the first position, is suction only (connected to the suction pipet). The second level, the second position, is suction and sprinkling (connected to both the pipet and the pump).

The delivery tube 15 and the aspiration pipet 29 may be located side-by-side or may be nested, with the delivery tube 15 located inside the aspiration pipet 29, as shown in FIG. 1A.

With reference to FIG. 1B, FIG. 1B depicts the auto-switch of the cylinder 1 of the present invention. The auto-switch 40 is a means that connects the first and second chambers 2 and 3 to either a vacuum source or the atmosphere alternately.

The following is the order of the change between vacuum and atmosphere in the first chambers 2: vacuum to atmosphere to vacuum to atmosphere. The following is the corresponding order of the change between vacuum and atmosphere in the second chamber 3: atmosphere to vacuum to atmosphere to vacuum. Within a chamber, the following occurs: vacuum to atmosphere to vacuum to atmosphere. These switches from the first and second chambers 2 and 3 and between vacuum and atmosphere are carried out automatically.

The auto-switch 40 is located between the first chamber 2 and the second chamber 3 at the interface of the separating wall 4 and the body cylinder wall. The auto-switch 40 comprises cylindrical tubing 30 that crosses the separating wall 4 with two opening ends, the first and second ports 9 and 10. A cylindrical body casing 31 longer than the cylindrical tubing 30, with both ends closed, but has two side holes, the first and second side holes 32 and 33, at each end. The first and second side holes 32 and 33 open to common opening 34 that is connected to the tube 35 leading to the vacuum source. The cylindrical body casing 31 also has first and second channels 36 and 37 at each end on the other side of the cylindrical body casing 31 to match the side openings 38 and 39 on the wall of the cylinder casing 1, near the separating wall 4, to communicate with the atmosphere.

Alternatively, the auto-switch 40 can be located in the center of the separating wall 4. In addition, the auto-switch could also be attached to, a part of, or incorporated with the rod 7.

The cylindrical body casing 31 slides into the cylindrical tubing 30. While the fit of the cylindrical body casing 31 inside the cylindrical tubing 30 is air tight, the cylindrical body casing 31 is still capable of shuttling from left to right. When the cylindrical body casing 31 slides completely into the cylindrical tubing 30 and both ends of the cylindrical body casing 31 and the cylindrical tubing 30 are lined up on one side such as the first chamber 2, on the other side, the second chamber 3, the other end of the cylindrical body casing 31 protrudes longer than the cylindrical tubing 30 so the cylindrical body casing 31 is exposed in the second chamber 3. During this configuration, in the first chamber 2, the first side hole 32 to the vacuum is closed, but the first channel 36 to the atmosphere is open. Also during this configuration, in the second chamber 3, the second side hole 33 to the vacuum is open to the second chamber 3, while the second channel 37 to the atmosphere is disconnected. As a result, when the vacuum controlled 17 connects the vacuum source to the auto-switch, the vacuum force will drive the second piston 6 to move toward the separating wall 4 with an accelerated rate. When the second piston 6 comes to the end and against the extended part of the cylindrical body casing 31, it will push the cylindrical body casing 31 so that now, the second chamber 3 is even and the first chamber 2 has the extended part of the cylindrical body casing 31. In the event the vacuum is applied to the first chamber 2, the opposite will result.

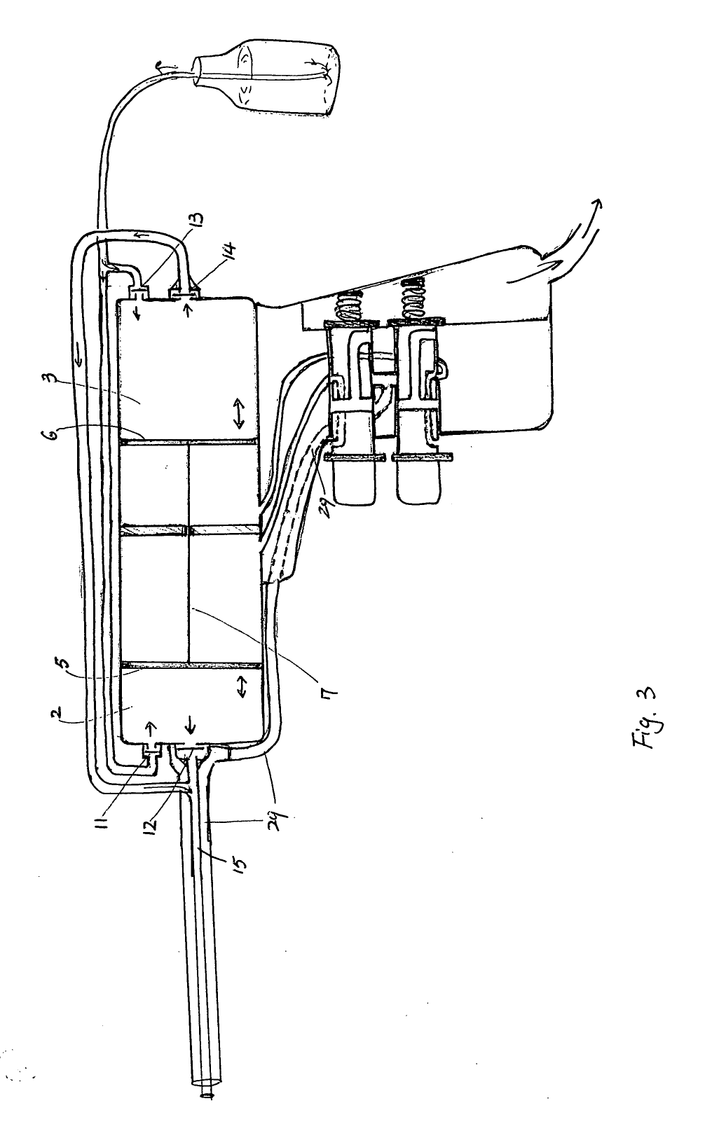

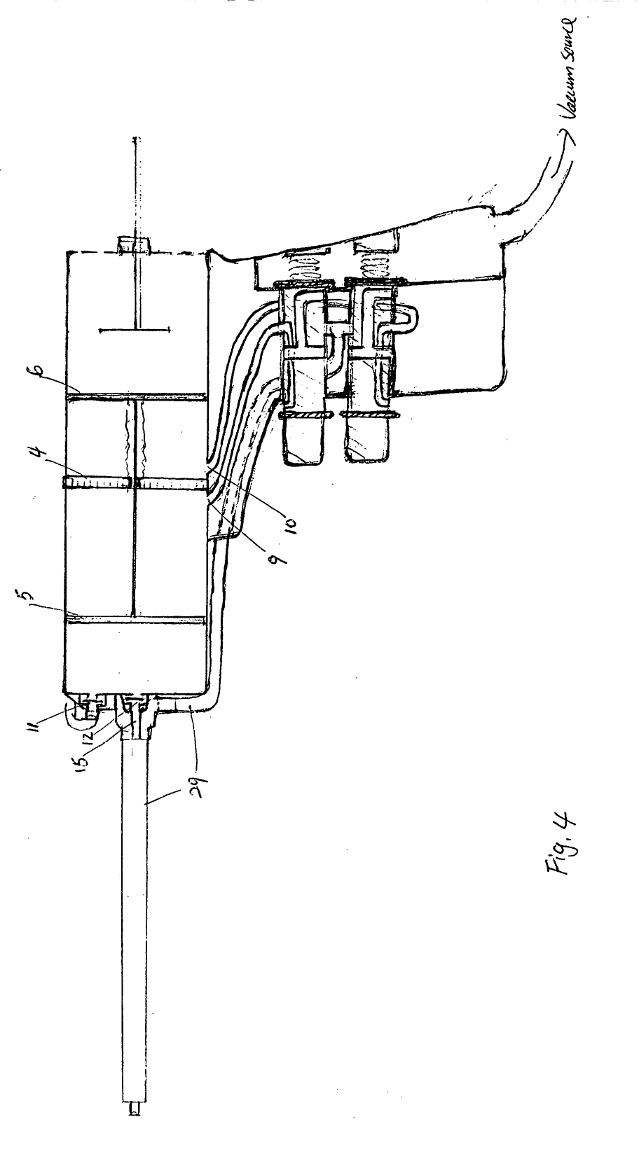

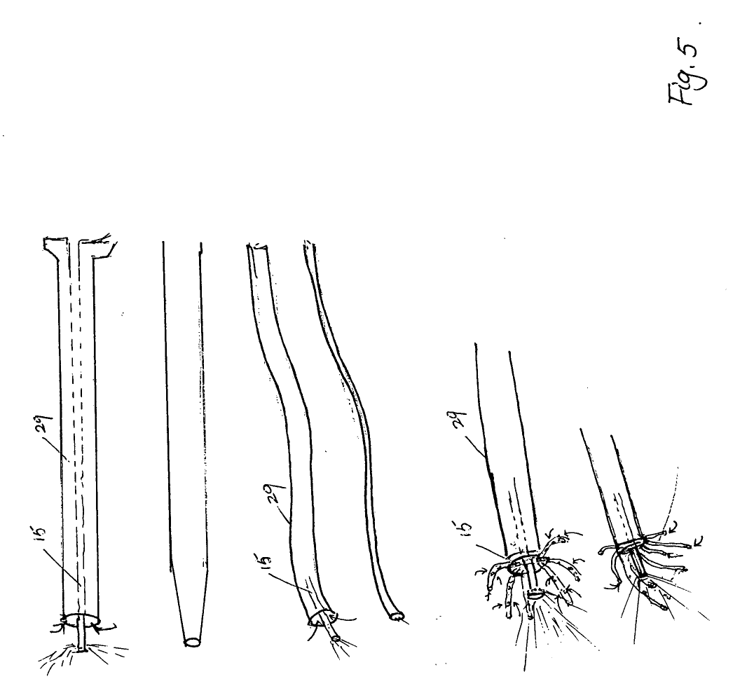

Alternate embodiments of the present invention are illustrated in FIGS. 2, 3, 4, and 5. Specifically FIG. 2 depicts an alternate embodiment of the present invention with the chambers and corresponding pistons set side by side, in parallel. FIG. 3 depicts an alternate embodiment of the present invention with different vacuum controllers, trumpet valves, and without the auto-switch. FIG. 4 depicts an alternate embodiment of the present invention with a single chamber and a volume setter (controller). FIG. 5 depicts various embodiments of probes/pipes for aspiration and fluid delivery connected to the pump of the present invention.

Thus, the present invention provides a lavage fluid pump that is lightweight, inexpensive, disposable, easy to make, easy to use, and without need of electric power of batteries. The present invention is low-noise, environmentally friendly, and cost-effective.

As such the method of making and using the device detailed above constitute the inventor's preferred embodiment and alternate embodiments to the invention. The inventor is aware that numerous configurations of the device as a whole or some of its constituent parts are available which would provide the desired results. While the invention has been described and illustrated with reference to specific embodiments, it is understood that these other embodiments may be resorted to without departing from the invention. Therefore the form of the invention set out above should be considered illustrative.

1. A vacuum driven pump comprising:
a cylinder casing;
a first chamber and a second chamber separated by a separating wall;
a first piston and a second piston linked by a rod which goes through said separating wall;
a first port associated with said first chamber and located between said separating wall and said first piston;
a second port associated with said second chamber and located between said separating wall and said second piston;
a first inlet valve to allow fluid to enter said first chamber and a second inlet valve to allow fluid to enter said second chamber wherein said first inlet valve and said second inlet valve are located at opposite ends of said cylinder casing;
a first outlet valve to allow fluid to exit said first chamber and a second outlet valve to allow fluid to exit said second chamber wherein said first outlet valve is located on the same end of said cylinder as said first inlet valve and said second outlet valve is located on the same end of said cylinder as said second inlet valve;
a vacuum source;
a liquid source; and
a vacuum controller.

2. The vacuum driven pump of claim 1 wherein said vacuum controller comprises:
a housing wherein said housing includes a first housing hole, a second housing hole, and a third housing hole;
a top trumpet valve wherein said top trumpet valve is cylindrical in structure, slides into said housing, includes a first, second, and third top holes and channels that match said first, second, and third housing holes, and is powered by a top spring; and
a bottom trumpet valve wherein said bottom trumpet valve is cylindrical in structure, slides into said housing, includes a first and second bottom holes and channels that match said first, second, and third housing holes, and is powered by a bottom spring.

3. The vacuum driven pump of claim 1 further comprising:
a delivery tube; and
an aspiration pipet.

4. The vacuum driven pump of claim 1 further comprising:
an elastic cushion placed around the hole to maintain the disconnection between said first chamber and said second chamber while allowing the free movement of said rod.

5. The vacuum driven pump of claim 1 further comprising:
a first flexible foldable bag located in said first chamber and a second flexible foldable bag located in said second chamber to enclose said rod and the link between said separating wall and said first piston and said second piston.

6. The vacuum driven pump of claim 1 further comprising an auto-switch located between said first chamber and said second chamber at the interface of said separating wall and the wall of said cylinder casing.

7. The vacuum driven pump of claim 6 wherein said auto-switch comprises:
a cylindrical tubing wherein said cylindrical tubing includes said first port and said second port, and crosses said separating wall;
a cylindrical body casing wherein said cylindrical body casing includes two closed ends, a first side hole, a second side hole, a first channel, a second channel, and is longer in length than said cylindrical tubing; wherein said first side hole and said second side hole open to a common opening; wherein said common opening is connected to a tube leading to a vacuum source.

8. The vacuum driven pump of claim 6 wherein said cylinder casing includes two side openings located proximal to said separating wall to match said first channel and said second channel.

9. The vacuum driven pump of claim 1 wherein said first port and said second port are located proximal to said wall.

10. The vacuum driven pump of claim 1 wherein said first port is connected to said vacuum source.

11. The vacuum driven pump of claim 1 wherein said first port is connected to the atmosphere.

12. The vacuum driven pump of claim 1 wherein said second port is connected to said vacuum source.

13. The vacuum driven pump of claim 1 wherein said second port is connected to the atmosphere.

14. A method for vacuum driving a lavage instrument comprising:
applying vacuum to a first chamber through a first port;
opening a second port of a second chamber to the atmosphere;
driving a first piston from one end of said first chamber to the other end of said first chamber;
simultaneously moving a second piston of said second chamber;
drawing fluid into the said first chamber through a first inlet valve via the movement of said first piston; and
expelling fluid out of said second chamber through a second outlet valve via the movement of said second piston.

15. A method for vacuum driving a lavage instrument comprising:
applying vacuum to a second chamber through a second port;
opening a first port of a first chamber to the atmosphere;
driving a second piston from one end of said second chamber to the other end of said second chamber;
simultaneously moving a first piston of said first chamber;
drawing fluid into the said second chamber through a second inlet valve via the movement of said second piston; and
expelling fluid out of said first chamber through a first outlet valve via the movement of said first piston.

16. A method for the continuous flow of fluid in and out of a vacuum driven pump for a lavage instrument comprising:
connecting a first outlet valve and a second outlet value to a delivery tube; and
connecting a first inlet valve and a second inlet valve to a liquid source.

17. A method for vacuum driving for a lavage instrument for suction purposes comprising:
pushing a bottom trumpet valve down until a first bottom hole and channel of said bottom trumpet valve aligns with a third housing hole;
connecting a vacuum source to an aspiration pipet.

18. The method of claim 17 for vacuum driving a lavage instrument for suction purposes and simultaneous sprinkling further comprising:
pushing a top trumpet valve down until a first top hole and channel of said top trumpet valve aligns with a first housing hole, and a second top hole and channel of said top trumpet valve aligns with a second housing hole;
communicating a vacuum source to an aspiration pipet and a tube of the driving force of a first piston and a second piston.

19. The vacuum driven pump of claim 1 wherein said first chamber is positioned to the left of said second chamber.

20. The vacuum driven pump of claim 1 wherein said first chamber is positioned above said second chamber, in parallel.

21. A vacuum driven pump comprising:
a cylinder casing;
a single chamber;
a volume controller;
a first piston and a second piston linked by a rod which goes through a hole in said separating wall;
a first port and a second port located in said first chamber and said second chamber respectively at either side of said first piston and said second piston respectively;
a first inlet valve to allow fluid to enter said first chamber and a second inlet valve to allow fluid to enter said second chamber wherein said first inlet valve and said second inlet valve are located at opposite ends of said cylinder casing;
a first outlet valve to allow fluid to exit said first chamber and a second outlet valve to allow fluid to exit said second chamber wherein said first outlet valve is located on the same end of said cylinder as said first inlet valve and said second outlet valve is located on the same end of said cylinder as said second inlet valve;
a vacuum source;
a liquid source; and
a vacuum controller.

22. The vacuum driven pump of claim 21 wherein said vacuum controller comprises:
a housing wherein said housing includes a first housing hole, a second housing hole, and a third housing hole;
a top trumpet valve wherein said top trumpet valve is cylindrical in structure, slides into said housing, includes a first, second, and third top holes and channels that match said first, second, and third housing holes, and is powered by a top spring; and
a bottom trumpet valve wherein said bottom trumpet valve is cylindrical in structure, slides into said housing, includes a first and second bottom holes and channels that match said first, second, and third housing holes, and is powered by a bottom spring.

23. The vacuum driven pump of claim 21 further comprising:
a delivery tube; and
an aspiration pipet.

24. A vacuum driven pump of the type that forms a first inlet valve and a second outlet valve, draws fluid in through said first inlet valve through the movement of a firs piston, and expels fluid out of said second outlet valve through the movement of a second piston, wherein:
said vacuum driven pump includes a sequence of a first chamber, a second chamber, separated by a separating wall, said first chamber being in communication with said first inlet valve and said first piston, and said second chamber being in communication with said second outlet valve and said second piston.
